(19) United States
(12) Patent Application Publication
(10) Pub. No.: US 2004/0167099 A1
(43) Pub. Date: Aug. 26, 2004

(54) Mucoadhesive tetracycline formulations

(75) Inventor: James Ronald Lawter, Yardley, PA (US)

Correspondence Address: PATREA L. PABST, PABST PATENT GROUP LLP, 400 COLONY SQUARE, SUITE 1200, ATLANTA, GA, 30361, US

(73) Assignee: OraPharma, Inc.

(21) Appl. No.: 10/679,913

(22) Filed: Oct. 6, 2003

Related U.S. Application Data
(60) Provisional application No. 60/416,742, filed on Oct. 7, 2002.

(51) Int. Cl. A61K 31/65; A61K 38/39

(52) U.S. Cl. 514/55; 514/152; 514/21

(57) ABSTRACT

Mucositis is treated and/or prevented by administrating to a patient a formulation containing a tetracycline and at least one cationic polymer and/or mucoadhesive material. The tetracycline may be in the form of a pharmaceutically acceptable salt or a base. The formulations may optionally also contain an antifungal agent to prevent fungal overgrowth due to reduction in the normal oral flora by the tetracycline. The formulation can be formed into liquid or solid dosage forms such as mouth rinse or tablet. Such compositions have the advantage of prolonged retention of the tetracycline in the mucosa of the oral cavity.

### CROSS-REFERENCE TO RELATED APPLICATIONS

This application claims benefit of U.S. Provisional Application No. 60/416,742, entitled “Mucoadhesive Tetracycline Formulations” to James R. Lawter, filed Oct. 7, 2002.

### FIELD OF THE INVENTION

The present application relates generally to formulations containing a tetracycline and at least one cationic polymer and/or mucoadhesive material that are especially useful for treating or preventing mucositis.

### BACKGROUND OF THE INVENTION

Mucositis is a dose-limiting side effect of cancer therapy and bone marrow transplantation and is not adequately managed by current treatment (Sonis, 1993a, “Oral Complications,” in: Cancer Medicine, pp. 2381-2388, Holand et al.; Eds., Lea and Febiger, Philadelphia; Sonis, 1993b, “Oral Complications in Cancer Therapy,” In: Principles and Practice of Oncology, pp. 2385-2394, De Vitta et al., Eds., J. B. Lippincott, Philadelphia). Oral mucositis is found in almost 100% of patients receiving radiotherapy for head and neck tumors, in about 40% of patients receiving chemotherapy, and in about 90% of children with leukemia (Sonis, 1993b, supra). Complications related to oral mucositis, though varying in the different patient populations, generally include pain, poor oral intake with consequent dehydration and weight loss, and systemic infection with organisms originating in the oral cavity leading to septicemia (Sonis, 1993b; U.S. Pat. No. 6,025,326 to Steinberg et al.). In addition to the oral cavity, mucositis may also affect other parts of the gastro-intestinal tract.

A variety of approaches to the treatment of oral mucositis and associated oral infections have been tested with limited success. For example, the use of an allopurinol mouthwash, an oral sucralfate slurry, and pentoxifyline were reported in preliminary studies to result in a decrease in mucositis. Rothwell and Spektor (Special Care in Dentistry, January-February 1990, pages 21-25) have shown that patients to whom an oral rinse containing tetracycline, diphenhydramine, nystatin, and hydrocortisone was administered developed less severe mucositis than patients receiving a control rinse. More recently, WO 99/45910 by Mucosal Therapeutics (Sonis and Fey) describes a method for treating and preventing mucositis by administering a non-steroidal anti-inflammatory (NSAID), an inflammatory cytokine inhibitor, or a mast cell inhibitor and second different therapeutic agent which is an NSAID, an inflammatory cytokine inhibitor, a mast cell inhibitor, a matrix metalloproteinase (MMP) inhibitor such as tetracycline or a nitric oxide inhibitor. A formulation including up to 1 mg/ml tetracycline is a particularly preferred formulation that has shown efficacy in animal models of radiation induced mucositis.

An improved tetracycline formulation for prevention or treatment of mucositis is described in WO 01/19362 by Orapharma. This application focuses on the utilization of a poorly absorbed tetracycline, which further helps in avoiding systemic side effects while preventing or minimizing the symptoms of mucositis. This application also discloses a stabilized tetracycline in the form of a polyvalent metal ion complex.

However, even though formulas are now available that are efficacious, there remains a need to produce formulations that are easier to formulate and more comfortable for the patient. For example, an oral rinse formulation of mecocycline must be prepared within 24 hours of use and kept in a refrigerator after preparation, since it is not stable in solution. Moreover, it is time consuming to prepare since it is made by adding water with much stirring to the drug, then adding buffer with more stirring to adjust pH, then administering.

It is therefore an object of the present invention to provide methods of making and using a composition to decrease the duration and/or severity of mucositis which is more stable to storage and/or easier to formulate and/or administer.

It is another object of the present invention to provide a method of making and using a composition to decrease the duration and/or severity of mucositis which has a prolonged retention in the mucosa of the oral cavity.

It is a further object of the present invention to provide a treatment that is safe, efficacious and easy for the patient to use.

### SUMMARY OF THE INVENTION

A formulation containing a tetracycline and at least one cationic polymer or a neutral polymer that becomes cationic upon contact with an aqueous medium such as saliva, mucoadhesive or gel forming material has been developed. The tetracycline may be in the form of a pharmaceutically acceptable salt or a base, in a crystalline or more preferably an amorphous form, or as a polyvalent metal ion complex of the tetracycline. The tetracycline can have either a good or a poor solubility in water. The tetracycline can be well absorbed or poorly absorbed tetracycline. The formulations may optionally contain other active ingredients, including anti-fungals, anti-inflammatories, antibiotics, and/or anesthetics.

The cationic polymer can be any pharmaceutically acceptable natural or synthetic polymer which has the desired physical or chemical properties to enhance retention in the mouth. Polymers will typically be cationic polymers, mucoadhesive polymers or polymers which form a gel or hydrogel that physically adheres to the mucosa. Preferably, the cationic polymer is a natural polymer such as gelatin or chitosan. Most synthetic polymers including a relatively high number of carboxylic groups will be mucoadhesive. Preferred polymers are biodegradable.

The formulation described herein can be a liquid dosage form as a solution or suspension of a pharmaceutically acceptable carrier or a solid dosage form. In one embodiment, the tetracycline can be formulated into a solid dosage form that forms a solution, suspension or hydrogel upon contact with an aqueous medium. In another embodiment, the solid dosage form is a compressed dosage form such as tablet that adheres to the mucosa even as it dissolves. The formulation may be designed for rapid release in the oral cavity, especially when administered under the tongue. The dosage forms can be prepared by any method suitable for making the different dosage forms described herein.

The composition described herein can be used to prevent or treat mucositis, especially mucositis resulting from radiation or chemotherapy for cancer. The method includes the step of administering to a patient an effective amount of a composition. The formulation may be administered prior to or after radiation or chemotherapy treatment is initiated, before or after symptoms of mucositis have developed.

### BRIEF DESCRIPTION OF DRAWING

FIG. 1 shows the relationship of fraction of ionized meclocycline versus pH.

### DETAILED DESCRIPTION OF THE INVENTION

I. Adherent Topical Tetracycline Formulations

Topical formulations for treating mucositis have been developed. These include as the active ingredient to treat the mucositis a tetracycline type compound, a cationic polymer or a neutral polymer that ionizes to form a cationic polymer, a mucoadhesive polymer and/or a gel forming material.

A. Tetracyclines

As used herein, tetracyclines include compounds that may or may not have antibiotic activity. The tetracyclines described herein can have high or poor water solubility and can be well absorbed or poorly absorbed. According to the FDA's Biopharmaceutics Classification System Guidance, a compound with high solubility is considered to be one where the highest dose is soluble in 250 ml or less of water over a pH range of 1 to 7.5. According to 21CFR 3020.33(e)(1) a compound with low solubility is one that has a solubility of less than 5 mg/ml. Preferred tetracyclines are those which are poorly absorbed when administered orally. Compounds which have bioavailibilities of about 50% or less are considered to be poorly absorbed according to 21 CFR 320.33(f)(2). The tetracycline may be one which is a salt or base of the drug, and may be crystalline or amorphous.

The tetracyclines are known to have pharmacological activities such as matrix metalloproteinase, nitric oxide synthetase and caspase inhibition that are independent of their antibiotic properties. These activities may be important in the treatment and prevention of mucositis. It is known that these pharmacological activities may be associated with tetracyclines that do not have significant antibiotic properties.

Tetracyclines are defined by the following structure:

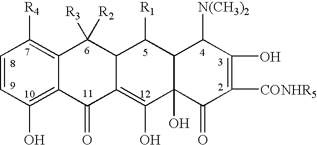

wherein R₁-R₅ are a hydrogen atom, a halogen atom, a hydroxyl group, or any other organic composition having 1-8 carbon atoms and optionally include a heteroatom such as nitrogen, oxygen, in linear, branched, or cyclic structural formats.

A wide range and diversity of embodiments within the definition of the above structure as are described within Essentials of Medicinal Chemistry John Wiley and Sons, Inc., 1976, pages 512-517. Preferably R₁ and R₂ are hydrogen or a hydroxyl group; R₃ is hydrogen or a methyl group; R₄ is a hydrogen atom, a halogen, or a nitrogen containing entity; and R₅ is a hydrogen atom, or nitrogen containing ring structure. The commonly known tetracycline analogues and derivatives include the following: oxytetracycline; chlortetracycline; demeclocycline; doxycycline; minocycline; rolitetracycline; lymecycline; sancycline; methacycline; apicycline; clomocycline; guamecycline; meglucycline; mepyclcline; penimepicycline; pipacycline; etocycline, penimocycline, and meclocycline.

Tetracycline derivatives that can be used as described herein, include tetracycline derivatives modified at positions 1 through 4 and 10 through 12, although these modifications may result in reduction in antibiotic properties, according to Mitscher, et al., J. Med. Chem. 21(5), 485-489 (1978). The configuration of the 4 carbon is important to the antibiotic properties of the tetracyclines. For the antibiotic tetracyclines, carbon 4 is in the S configuration. The 4-epimers of the tetracyclines, which have the R configuration at the 4 carbon, have significantly reduced antibiotic activity. Other such non-antibiotic tetracycline analogs include the 4-de(dimethylamino) derivatives of the tetracyclines listed in the above paragraph. Specific examples include: 6-demethyl-6-deoxy-4-dedimethylaminotetracycline; 6-demethyl-6-deoxy-4-dedimethylamino-7-dimethylaminotetracycline; 6-demethyl-6-deoxy-4-dedimethylamino-7-chloro-tetracycline; 4-hydroxy-4-dedimethylaminotetracycline; 6a-deoxy-5-hydroxy-4-dedimethylaminotetracycline; 4-dedimethylamino-5-oxytetracycline, and 4-dedimethylamino-11-hydroxy-12a-deoxytetracycline. Further examples of tetracyclines with reduced antibiotic activity include 6-α-benzylthiomethylenetetracycline, 6-fluoro-6-demethyltetracycline, and 11α-chlorotetracycline.

In one preferred embodiment, the tetracycline is meclocycline.

Other tetracycline related compounds that can be used as described herein are the 9-((substituted)amido)tetracyclines. The latter include the compounds described in U.S. Pat. Nos. 5,886,175, 5,284,963, 5,328,902, 5,386,041, 5,401,729, 5,420,272, and 5,430,162. Specifically, the 9-((substituted)amido)tetracycline may be 9-(t-butylglycylamido)-minocycline.

Preferred poorly absorbed tetracyclines include compounds of the following structure:

wherein R¹, R², R³, R⁴, R⁵, R⁶, R⁷, and R⁸ can be H, C1-C3 alkyl, phenyl,

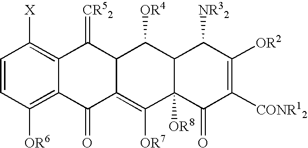

and aryl groups; and

wherein X is an H, alkyl, alkoxy, phenoxy, aryloxy, amino group, amide, acyl, and

halo group; and pharmaceutically acceptable salts thereof.

The most preferred compound of this general structure is

wherein R¹, R², R⁵, R⁶, R⁷, and R⁸ are H;

wherein R³ is CH₃; and

wherein X is a chloro group. The generic name for this compound is meclocycline.

The preparation of meclocycline and its analogs and derivatives are known. For example, U.S. Pat. No. 3,966,808 to Luciano discloses methods for manufacturing 6-methylenetetracyclines.

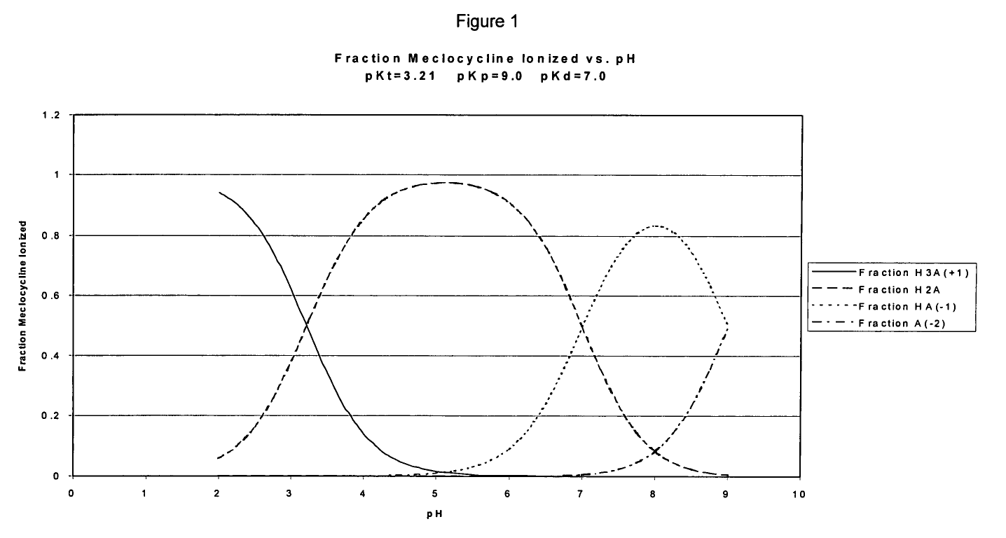

As FIG. 1 shows, tetracycline ionizes in response to pH. At a low pH, for example pH=2, the predominant form of tetracycline is cationic tetracycline. At a higher pH, for example pH=7 or above, the predominant form is anionic tetracycline.

B. Cationic Polymers, Mucoadhesive Polymers, Gel Forming Polymers

Cationic polymers include chitosan and other natural polymers, such as gelatin, with high isoelectric points that are positively charged at the pH of the oral cavity. Acid treated gelatins have isoelectric points in the desired range. Fish gelatin is particularly advantageous, since aqueous solutions are liquid at room temperature. Also there is no concern about transmissible spongiform encephalopathy with fish gelatin as there is with bovine sourced gelatin.

Two other classes of polymers that generally show useful bioadhesive properties are hydrophilic polymers and hydrogels. In the large class of hydrophilic polymers, those containing carboxylic groups (e.g., poly[acrylic acid]) exhibit the best bioadhesive properties. Some of these materials are water-soluble, while others are hydrogels.

Representative natural polymers include proteins, such as zein, modified zein, casein, gelatin, gluten, chitosan or collagen, and polysaccharides, such as cellulose, dextrans, polyhyaluronic acid, and alginic acid.

Representative synthetic polymers include poly(vinyl alcohols), polyacrylamides, polyalkylene glycols, polyalkylene oxides, polyvinyl esters, polyvinylpyrrolidone, and copolymers thereof. Synthetically modified natural polymers include alkyl celluloses, hydroxyalkyl celluloses, cellulose ethers, and cellulose esters. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(ethylene glycol), poly(ethylene oxide), poly(vinyl acetate), polyvinyl pyrrolidone, and polyvinylphenol. These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

In some instances, a polymeric material can be modified to improve bioadhesion. For example, the polymers can be modified by increasing the number of carboxylic groups accessible during biodegradation, or on the polymer surface. The polymers can also be modified by binding amino groups to the polymer. Alternatively, the polymers can be modified using any of a number of different coupling chemistries that covalently attach ligand molecules with bioadhesive properties.

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of bioadhesive ligands. Any polymer that can be modified through the attachment of lectins can be used as a bioadhesive polymer.

Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range would yield chains of 120 to 425 amino acid residues attached to the polymer. The polyamino chains would increase bioadhesion by means of chain entanglement in mucin strands as well as by increased carboxylic charge.

C. Pharmaceutically Acceptable Carriers or Fillers

Carriers for liquid formulations

The formulations may be prepared as a liquid, semi-solid, or solid. In a liquid formulation, these compositions contain about 0.001 to 1 mg/ml of the tetracycline. In a solid formulation such as tablet, these compositions contain preferably 0.1-100 mg, most preferably 1 to 10 mg tetracycline. The tetracycline to polymer weight ratio may vary from 1 to 0.1 to 1 to 100. Preferably the ratio ranges from 1 to 1 up to 1 to 10.

The compositions are topically administered to the oral mucosa and then swallowed or spit out. Formulation types suitable for this route of administration include liquids applied as mouth rinses; solid dosage forms that may dissolve in the mouth; and semisolids that may be applied to oral cavity surfaces.

Tetracyclines in general may not be sufficiently stable in aqueous solutions to permit formulations with long shelf lives at room temperature, i.e. a year or more, to be prepared. Stability of the tetracyclines varies greatly with structure. However, solids for re-constitution as aqueous based solutions prepared either by the patient or by a pharmacist prior to administration to the patient can be used, even for the least stable members of the class. Also polyvalent metal ion complexes may be prepared that are stable in contact with water at room temperature for two years or more. Examples are the calcium and magnesium organic or inorganic salts or complexes. These salts or complexes may be suspensions in water.

The stability of the tetracyclines in aqueous solutions is pH dependent. Procedures for choosing the optimum pH and buffering agents are well known. Other factors that affect stability in solution are also well known. For example, antioxidants may be added to reduce the rate of degradation due to oxidation.

In addition to the tetracycline and antifungal agents, an aqueous liquid preparation may contain buffers, surfactants, humectants, preservatives, flavorings, stabilizers (including antioxidants), colorants, and other additives used in preparations administered into the oral cavity.

The compositions used as mouthwashes preferably should have a pH of 3.5 to 8. A preparation having a pH of less than about 4 would be likely to cause a stinging sensation. Furthermore, the preparations having a higher pH are often unpleasant to use. The active agents need not be in solution to be effective. The active agents may be present wholly or in part as suspensions in a pharmacologically acceptable carrier, for example, water or an alcohol.

Generally, a water solution of tetracycline has a pH in the weak acidic range, e.g., pH 4-6. The preparations are buffered as necessary to provide the appropriate pH range, for example pH 6.5-9.0. For mouth rinse formulation, the preferred pH range is pH 7.8-8.0. Appropriate buffer systems include citrate, acetate, tromethamine, bicarbonates and benzoate systems. Preferably, the buffer system is tromethamine, which has a pKa of in the range of pKa 8-9. However, any buffer system commonly used for preparing medicinal compositions would be appropriate. While the vehicle used generally is primarily water, other vehicles may be present such as alcohols, glycols (polyethylene glycol or polypropylene glycol are examples), glycerin, and the like may be used to solubilize the active agents. Surfactants may include anionic, nonionic, amphoteric and cationic surfactants, which are known in the art as appropriate ingredients for mouthwashes.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, gellan gums, and gelatin. High viscosity formulations may cause nausea in chemotherapy and radiation patients and are therefore not preferred. Gelatin or its derivatives are preferred as viscosity modifying agents. Gellan gums are also preferred modifying agents since aqueous solutions containing certain gellan gums may be prepared so that they will experience an increase in viscosity upon contact with electrolytes. Saliva contains electrolytes that will interact with such a gellan containing solution so as to increase their viscosity. The increased viscosity will promote retention of the solutions in the oral cavity and provide greater effectiveness due to increased contact time with the affected tissues.

Flavorings used in the mouth rinse art such as peppermint, citrus flavorings, berry flavorings, vanilla, cinnamon, and sweeteners, either natural or artificial, may be used. Flavorings that are known to increase salivary electrolyte concentrations may be added to increase the magnitude of the viscosity change.

In order to improve the patient acceptability, it is desirable to add an appropriate coloring and/or flavoring material. Any pharmaceutically acceptable coloring or flavoring material may be used.

Additional antimicrobial preservatives may be component of the formulation in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

Fillers for solid dosages

Pharmaceutically acceptable fillers and excipients can be used to formulate the tetracyclines described herein into solid dosage forms. Suitable solid dosage forms include powders or tablets that are designed for constitution as solutions by dissolution or suspension in a liquid vehicle and include troches, pastilles or lozenges that dissolve slowly in the mouth. In one preferred embodiment, the solid dosage form is tablet.

For convenience of use, solids designed to be dissolved to prepare a liquid dosage form prior to administration preferably are rapidly dissolving. Technologies to produce rapidly dissolving solids are well known in the art. These include spray-drying, freeze-drying, particle size reduction, inclusion of effervescent components and optimizing the pH of the dissolution medium.

Additional excipients generally known in the art can be used to formulate the tetracyclines into a suitable dosage form (see, for example, Encyclopedia of Controlled Drug Delivery, Edith Mathiowitz, Ed., John Wiley & Sons, Inc., New York, 1999; and U.S. Pat. No. 5,558,880, the teachings of which and references cited therein are incorporated herewith by reference). For example, for a solid dosage form such as tablet prepared by a freeze-drying process, sugars such as lactose and/or mannitol or the derivatives thereof can be used in the formulation.

One general requirement for the solid dosage form is that the tetracycline can rapidly dissolve on contact with water. The solubilities of tetracyclines are a function of pH since they have several ionizable functional groups. Tetracyclines generally have a minimum in their pH-solubility curves between a pH of 3 and 6. The rate of dissolution of acidic salts may be increased by dissolving in a neutral to basic buffer. Dispersal of such salts may optimally be done at low pH.

Various solid dosage forms, the materials making the solid dosage forms, and methods for making the solid dosage forms have been documented. For example, U.S. Pat. Nos. 6,316,027; 5,648,093; and 4,754,597 disclose fast dissolving dosage forms of a drug and the process of making the dosage forms. U.S. Pat. Nos. 6,156,339; 5,837,287; 5,827,541 describe methods for the preparation of solid rapidly disintegrating dosage forms of a drug. Various forms of blister pack and the method of making the pack or the blister pack form of a drug has been described in, for example, U.S. Pat. Nos. 5,729,958; 5,046,618; 5,343,672; and 5,358,118. U.S. Pat. No. 5,631,023 discloses rapidly dispersing pharmaceutical tablets of a drug. U.S. Pat. No. 5,558,880 discloses a fast dissolving, solid dosage form formed of a matrix containing gelatin, pectin and/or soy fiber protein. U.S. Pat. No. 5,188,825 describes using an ion exchange resin to bond a water soluble active agent so as to form a substantially water insoluble complex.

In one embodiment, the tetracycline can be formulated into a solid dosage form that forms a solution upon contact with an aqueous medium. The dosage form includes a tetracycline and a buffer which disintegrates in the aqueous medium within two minutes to form a solution with a pH greater than 5. In one embodiment, the aqueous medium is saliva. In another embodiment, the aqueous medium is water in a volume of, for example, 10 ml, that rapidly dissolves the solid dosage to form a mouth rinse in situ.

In another embodiment, the solid dosage form is a hard, compressed dosage form such as tablet that is rapidly dissolvable upon contact with an aqueous medium. The hard, compressed dosage includes a tetracycline and a matrix including a direct compression filler and a lubricant. The dosage form is adapted to rapidly dissolve in the mouth of a patient and thereby liberate the tetracycline. The hard, compressed dosage has a friability of, for example, about 2% or less when tested according the USP. The dosage form has a hardness of at least about 15 Newtons or higher. Hard, compressed dosage forms have been described, for example, in U.S. Pat. Nos. 6,221,392; 6,024,981; and 5,576,014, the teachings of which have been fully incorporated herein by reference.

In still another embodiment, the formulation described herein is a solid dosage form that includes a tetracycline which disintegrates within a short period, preferably two minutes, when placed in an aqueous medium to form a suspension or paste which slowly releases the tetracycline. The aqueous medium can be saliva or water. Preferably, the tetracycline is released over a period of two minutes or longer when placed in the aqueous medium.

In still another embodiment, the formulation described herein is a solid dosage form that includes a polyvalent metal ion complex of a tetracycline. The dosage form disintegrates within a short period, preferably, two minutes, when placed in an aqueous medium to form a suspension or paste containing the tetracycline. The aqueous medium can be saliva or water. Preferably, the tetracycline is released over a period of two minutes or longer when placed in the aqueous medium.

In still another embodiment, the formulation described herein is a solid pharmaceutical dosage form that includes a tetracycline and a water-soluble or water dispersible carrier adapted for dissolution in the oral cavity over a period of more than two minutes.

D. Other Active Agents

Other medicinal agents may be added for purposes of alleviating other undesirable conditions in the mouth. Such agents may include, for example, local anesthetics, antibacterial agents, and emollients, as well as anti-fungal agents.

Anti-Fungal Agents

Antibiotic tetracyclines applied topically in the oral cavity may reduce the number of susceptible flora to such an extent that competitive conditions that hold non-susceptible organisms in check may not be effective. In particular, fungi, which are not susceptible to tetracyclines, may increase drastically in number. To avoid this, an antifungal agent may be added to the composition. Examples of antifungal agents that have been shown to be effective in preventing or treating fungal overgrowth are nystatin and clotrimazole. These agents may be added to a liquid tetracycline dosage form as a powder to form a suspension. The approved dosage for Clotrimazole, 10 mg is three times a day for mucositis. The approved dosage of Nystatin is 200,000 to 400,000 units, 4 to 5 times a day for up to 14 days in pastilles.

Examples of local anesthetics are lidocaine and a eutectic mixture of lidocaine and prilocaine. Lidocaine is administered in solution at a concentration of 2%, at a dose of 15 ml, at intervals of not less than three hours. The eutectic mixture is equimolar, administered at a total concentration of up to 5%. Either could be incorporated in an aerosol at similar doses.

II. Process of Preparing the Formulation

The topical formulation can be prepared according to the dosage form of the formulation. Liquid dosage forms can be prepared by, for example, admixing tetracycline and other ingredients. Various methods for making solid dosage forms of a drug have been described in, for example, U.S. Pat. Nos. 6,316,027; 5,648,093; 4,754,597; 6,156,339; 5,837,287; 5,827,541; 5,729,958; 5,046,618; 5,343,672; 5,358,118; 5,631,023; 5,558,880; 5,188,825; 6,221,392; 6,024,981; and 5,576,014, the teachings of which are fully incorporated herein by reference.

The preparation of solid dosage forms varies with the particular form of the solid dosage. In one embodiment, the process involves the following steps: (i) preparing a solution of a water-soluble or water dispersible carrier, a filler, and the tetracycline; (ii) forming discrete units of the solution; and (iii) removing the solvent from the discrete units under vacuum thereby forming solid dosage forms containing a network of carrier/filler carrying a dose of the tetracycline.

In another embodiment, the process of making a solid dosage form involves: (i) preparing a suspension that includes water, a water-soluble or water dispersible carrier, a filler, and the tetracycline, a part of which is present as a suspension of solid particles; (ii) forming discrete units of the suspension and (iii) removing the solvent from the discrete units under vacuum thereby forming solid dosage forms that include a network of carrier/filler carrying a dose of the tetracycline.

In still another embodiment, the process of making a solid dosage form involves: (i) preparing a mixture including water, a water-soluble or water dispersible carrier, a filler, and the tetracycline in the form of a polyvalent metal complex; (ii) forming discrete units of the mixture; and (iii) removing the solvent from the discrete units under vacuum thereby forming solid dosage forms that include a network of carrier/filler carrying a dose of the tetracycline.

III. Methods of Treatment

Methods of using the formulations disclosed herein generally involve applying the formulations topically to mucosal surfaces of the oral cavity and gastrointestinal tract. One to six applications per day beginning 24 hours before chemotherapy or radiation until conclusion of treatment are made. The typical volume of a mouthwash would be between 5-15 ml, preferably about 10.0 ml.

Therapy is continued for as long as the patient is receiving radiation or chemotherapy.

In one embodiment, the method for treating or preventing oral mucositis resulting from radiation or chemotherapy for cancer. The method includes the step of administering to a patient an effective amount of a liquid formed by placing one of the solid dosage form described herein in an aqueous solution. The liquid is administered as, for example, a mouth-rinse.

In another embodiment, the method for treating or preventing oral mucositis resulting from radiation or chemotherapy for cancer includes the step of administering a solid dosage form described herein to the oral cavity of a patient, preferably sublingually, wherein the tetracycline is released.

The present invention will be further understood by reference to the following non-limiting examples.

Methods and Materials

The following animal model was used to demonstrate the effectiveness of the poorly absorbed tetracyclines in treating mucositis.

Hamsters were randomly assigned to treatment groups with eight (8) animals per group. Each group was treated either with a drug solution or a control, water.

Animals were dosed three times a day for 22 days. The first dose was applied on day −1. Either a solution of the drug or water alone was applied in a volume of 0.1 ml three times per day.

Mucositis was induced by acute radiation exposure of the check pouch. A single dose of radiation (35 Gy/dose) was administered to all animals on Day 0. Prior to irradiation, animals were anesthetized with an intraperiotoneal injection of sodium pentobarbital (80 mg/kg) and the left buccal pouch was everted, fixed and isolated using a lead shield.

Beginning on day 6 and continuing every other day up to day 28, the cheek pouch was photographed. On days that photographs were taken, prior to the first dosing of the day, the animals were anesthetized using an inhalation anesthetic and the left cheek pouch of each animal was rinsed vigorously with sterile water to remove residual food debris or foreign contamination and blotted dry with a gauze sponge. The appearance of the cheek pouch was scored visually by comparison to a validated photographic scale, ranging from 0 for normal to 5 for severe ulceration (clinical scoring). In descriptive terms, this scale is defined as follows:

Score Description

0 Pouch completely healthy. No erythema or vasodilatation

1 Light to severe erythema and vasodilatation. No erosion of mucosa

2 Severe erythema and vasodilatation. Erosion of superficial aspects of mucosa leaving denuded areas. Decreased stippling of mucosa

3 Formation of off-white ulcers in one or more places. Ulcers may have a yellow/gray color due to pseudomembrane formation. Cumulative size of ulcers up to ¼ of the pouch surface. Severe erythema and vasodilatation

4 Cumulative size of ulcers ¼ to ½ of the pouch surface. Loss of pliability. Severe erythema and vasodilatation

5 Virtually all of pouch is ulcerated. Loss of pliability (pouch can only partially be extracted from mouth).

A score of 1-2 represents mild stage of the disease, whereas a score of 3-5 indicates moderate to severe mucositis.

### EXAMPLE 1

### Freeze-Dried Meclocycline Gellan Gum Formulations

Meclocycline hydrochloride powder formed by freeze drying in bulk is added to a solution containing gellan gum at a concentration of 0.5 mg/ml. The tetracycline concentration is 0.1 mg/ml. The solution also contains methyl and propyl parabens as antimicrobial preservatives at concentrations of 0.18% and 0.02%, respectively and tromethamine buffer.

### EXAMPLE 2

### Miconized Meclocycline Gellan Gum Buffered Formulations

Meclocycline hydrochloride powder formed by micronization is added to a solution containing gellan gum at a concentration of 0.5 mg/ml. The tetracycline concentration is 0.05 mg/ml. The solution also contains methyl and propyl parabens as antimicrobial preservatives at concentrations of 0.18% and 0.02%, respectively and tromethamine buffer.

### EXAMPLE 3

### Spray-Dried Meclocycline Gellan Gum Formulation

Meclocycline hydrochloride powder formed by spray drying is added to a solution containing gellan gum at a concentration of 0.5 mg/ml. The tetracycline concentration is 0.01 mg/ml. The solution also contains methyl and propyl parabens as antimicrobial preservatives at concentrations of 0.18% and 0.02%, respectively and tromethamine buffer.

### EXAMPLE 4

### Meclocycline Suspension

A suspension of meclocycline sulfosalicylate is formed by addition of micronized drug to an aqueous solution containing 0.5% gellan gum and methyl and propyl parabens as antimicrobial preservative.

### EXAMPLE 5

### Meclocycline Sulfosalicylate Suspension

A suspension of meclocycline sulfosalicylate is formed by addition of micronized drug to a unit dose quantity of an aqueous solution containing 0.5% gellan gum. No antimicrobial preservative is required since the formulation is used immediately after preparation.

### EXAMPLE 6

### Meclocycline Sulfosalicylate Effervescent Tablet

Compress mixture comprised of 7.9 mg meclocycline sulfosalicylate, 10 mg gelatin, 20 mg mannitol, 31.2 mg microcrystalline cellulose, 20 mg sodium bicarbonate, 10 mg citric acid (anhydrous), 0.5 mg magnesium stearate and 0.4 mg colloidal silicone dioxide (total tablet weight 100 mg) in a dry atmosphere.

### EXAMPLE 7

### Meclocycline Base Freeze-Dried Tablet

Disperse 20 mg/mL meclocycline base in a cold solution containing 40 mg/mL gelatin and 30 mg/mL mannitol, fill pre-formed unit dose wells with the liquid mixture, freeze-dry and apply lid to well.

### EXAMPLE 8

### Meclocycline Sulfosalicylate Freeze-Dried Tablet

Disperse 6.3 mg/mL meclocycline sulfosalicylate in a cold solution containing 40 mg/mL gelatin and 30 mg/mL mannitol, fill pre-formed unit dose wells with the liquid mixture, freeze dry and apply lid to well.

Those skilled in the art will recognize, or be able to ascertain using no more than routine experimentation, many equivalents to the specific embodiments of the present application described herein. Such equivalents are intended to be encompassed by the following claims.

I claim:

1. A composition for application to the oral mucosa comprising a tetracycline and a pharmaceutically acceptable carrier selected from the group consisting of a mucoadhesive polymer, a viscous polymer gel and a hydrogel.

2. The composition of claim 1 wherein the mucoadhesive polymer is a cationic polymer.

3. The composition of claim 1 wherein the polymer is a natural polymer.

4. The composition of claim 1 wherein the tetracycline is poorly absorbed.

5. The composition of claim 1 wherein the tetracycline is meclocycline.

6. The composition of claim 1 wherein the tetracycline is amorphous.

7. The composition of claim 1 wherein the tetracycline is a base.

8. The composition of claim 1 wherein the tetracycline is a salt.

9. The composition of claim 1 for treating or preventing oral mucositis comprising an effective amount of tetracycline to treat mucositis.

10. The composition of claim 1 wherein the mucoadhesive polymer ionizes to form a cationic polymer upon contact with an aqueous medium.

11. The composition of claim 1 wherein the mucoadhesive polymer is a polyamine.

12. The composition of claim 1 wherein the carrier provides sustained or controlled release of the tetracycline.

13. The composition of claim 2 wherein the cationic polymer is chitosan.

14. The composition of claim 1 wherein the mucoadhesive polymer is gelatin

15. The composition of claim 2 wherein the cationic polymer is a gelatin with an isoelectric point of 7 or more.

16. The composition of claim 14 wherein the gelatin is fish gelatin.

17. The composition of claim 1 wherein the hydrogel carrier provides for rapid release of the tetracycline.

18. A method for treating or preventing oral mucositis resulting from radiation or chemotherapy for cancer comprising administering to a patient an effective amount of a composition comprising a tetracycline and a pharmaceutically acceptable carrier selected from the group consisting of a mucoadhesive polymer, a viscous polymer gel and a hydrogel.
